(19) United States
(12) Patent Application Publication
(10) Pub. No.: US 2022/0064089 A1
(43) Pub. Date: Mar. 3, 2022

(54) METHOD FOR PRODUCING 1,2-ALKANE DIOLS IN A SOLID DOSAGE FORM

(71) Applicant: SYMRISE AG, Holzminden (DE)

(72) Inventors: Jürgen SIEWERT, Rollshausen (DE); Torsten STEPHAN, Holzminden (DE)

(21) Appl. No.: 17/422,947

(22) PCT Filed: Jan. 23, 2019
(86) PCT No.: PCT/EP2019/051575

§ 371 Date: Jul. 14, 2021

(51) Int. Cl. C07C 31/20; C07C 29/78

(52) U.S. Cl. CPC C07C 31/20; C07B 2200/13; C07C 29/78

(57) ABSTRACT

A process is proposed for producing 1,2-alkanediols in a solid dosing form, comprising or consisting of the following steps:
(a) providing a melt of at least one 1,2-alkanediol;
(b) cooling the melt from step (a) to a temperature below the melting point of the 1,2-alkanediol or of the 1,2-alkanediol mixture to obtain a prescratched melt consisting of a supercooled 1,2-alkanediol or a supercooled 1,2-alkanediol mixture having a content of 1,2-alkanediol seed crystals;
(c) contacting the mixture from step (b) with a cooled surface to obtain 1,2-alkanediols or 1,2-alkanediol mixtures in crystalline form.

### FIELD OF THE INVENTION

The invention is in the field of pelletization technology and relates to an improved process for producing 1,2-alkanediols in a solid dosing form.

### TECHNOLOGICAL BACKGROUND

1,2-Alkanediols have in recent years been increasingly used as emulsion-forming constituents in a wide variety of applications, these applications ranging from personal care products to industrial applications in the field of printer inks.

Whereas the lower 1,2-alkanediols (propane-1,2-diol to hexane-1,2-diol) have a liquid state of aggregation at room temperature, the middle to higher 1,2-alkanediols (from C8=octane-1,2-diol upwards) are solids at room temperature. The melting points of the solids become higher with increasing chain length:

Octane-1,2-diol: 27 to 31° C.
Decane-1,2-diol: 45 to 48° C.
Dodecane-1,2-diol: 56 to 60° C.
Tetradecane-1,2-diol: 64 to 70° C.

Melts of pure C8 to C14 alkanediols (>95% alkanediol content) show only a slight tendency to solidify after cooling. The materials appear to remain in a metastable, liquid state for hours or days until seed cells for solidification/crystallization have formed. Once such seed cells have formed, crystallization then occurs which, depending on the temperature gradient, is overlaid by disorderly solidification of the melt. After the product has solidified, a partially crystalline product is thus obtained.

The solidification process of the materials usually takes place with volume effects, i.e. the material undergoes an expansion in volume, the solid material having a rough, uneven surface. From a production viewpoint, this material property is problematic, since, in order to avoid deformation of the packaging, filled containers cannot be closed until after solidification has taken place. To reduce or avoid delays in material flow, rapidly solidifying melts are desirable.

The use/dosing of solids for the preparation of application mixtures typically represents a major technical and logistical effort. The solids must be melted in the delivered containers (typically up to 180 kg in steel drums) in order for them to then be dosed into the mixture in liquid form. The melting process needs to take place here under an inert gas atmosphere, since damage to the product cannot be ruled out in the event of exposure to atmospheric oxygen at elevated temperatures. A melting process of this kind can take up to several hours or even days, depending on the technical options available to the user. Short-term production planning is therefore possible only to a limited degree. In addition, an inert gas atmosphere must likewise be ensured for the cooling process, in order to rule out damage to the product. This is particularly important when only small amounts of alkanediols are used for a mixture and the storage containers need to be warmed up/cooled down with corresponding frequency.

### RELEVANT PRIOR ART

WO 2016/016154 A1 (SYMRISE) discloses a process for producing solid coolants, in which a prescratched melt of menthol compounds is applied by uniform dropletization onto a precooled surface.

### OBJECT OF THE INVENTION

The above-described problems in the production of mixtures using small amounts of alkanediols give rise to the need for a solid dosing form for 1,2-alkanediols that allows dosing without prior melting.

The material characteristics described above mean that direct solidification of the melt of medium-chain 1,2-alkanediols by dropletization or pelletizing, the production of flakes (using a flaking roller), or crystallization is not possible. When dropletizing e.g. decane-1,2-diol, pellets could not be obtained, all that could be obtained being a thin product film. After application of the melt in droplet form onto a cooled surface, the solid decane-1,2-diol was present after 2 minutes. Detachment from the cooled surface proved difficult—the solidified/flaked material could be obtained only alongside the formation of fines (fragments). Furthermore, the waxy portion of the solid obtained underwent delayed crystallization, with a rough, brittle surface becoming evident on the solid after a few days. On storage of the flakes thus obtained, mechanical friction results in a not inconsiderable fines fraction in the packaging material or—if delayed crystallization is not yet fully complete—leads to the material forming lumps. The processes just described promote the formation of both fines and coarse fractions, which are undesirable and disadvantageous for later dosing of solids. Lumpy aggregates sometimes have to be split apart before use at considerable mechanical effort, while fines can be handled only with increased levels of protection.

As previously described, the 1,2-alkanediol solid obtained by direct solidification/dropletization is of only limited practical use. This accordingly gives rise to a need for alternative production processes for solid dosing forms.

The object of the present invention was therefore to provide an improved process for producing solid dosing forms of 1,2-alkanediols that is free of the disadvantages outlined in the introduction. In particular, the process should be characterized by pellets having a very low fine dust content, the smoothest possible surface even after prolonged storage, and the absence of any tendency to form lumps.

### DESCRIPTION OF THE INVENTION

The invention firstly provides a process for producing 1,2-alkanediols in a solid dosing form, comprising or consisting of the following steps:
(a) providing a melt of at least one 1,2-alkanediol;
(b) cooling the melt from step (a) to a temperature below the melting point of the 1,2-alkanediol or of the 1,2-alkanediol mixture to obtain a prescratched melt consisting of a supercooled 1,2-alkanediol or a supercooled 1,2-alkanediol mixture having a content of 1,2-alkanediol seed crystals;
(c) contacting the mixture from step (b) with a cooled surface to obtain 1,2-alkanediols or 1,2-alkanediol mixtures in crystalline form.

In the search for solutions to the problem, the dropletization/pelletization of prescratched melts of 1,2-alkanediols was investigated. For this, the melts were cooled, with stirring, in a jacketed vessel having a wall-sweeping paddle stirrer. In this process, seed crystals were generated, and at the same time crushed, by the wall-sweeping stirrer. Thermostatic cooling below the melting point of the 1,2-alkanediol allowed a mixture/slurry/paste of seed crystals and the supercooled diol to be produced. It was surprisingly found that such mixtures could be simply dropletized into pellets. For this, the crystal paste was dripped onto a cooled surface by means of a pipette; the pellets thus obtained were cooled further at room temperature for a certain time and could then be easily removed from the cooled surface with a scraper.

The pellets thus obtained have a smooth to slightly rough surface, even after prolonged storage. The higher the proportion of crystals in the processed slurry, the smoother the surfaces in appearance. Even after prolonged storage of the freshly produced pellets, no formation of lumps was observed in the pellets.

### 1,2-Alkanediols

1,2-Alkanediols having 8 to 14 carbon atoms are used according to the invention. At least one 1,2-alkanediol and preferably at least two alkanediols selected from the group formed from octane-1,2-diol, decane-1,2-diol, dodecane-1,2-diol, and tetradecane-1,2-diol are used here. Normally, technical grade 1,2-alkanediols having a 1,2-alkanediol content of at least 92% by weight, preferably at least 95% by weight, and in particular at least 98% by weight, are used.

In principle, it is also possible for the mentioned medium-chain 1,2-alkanediols to have shorter-chain homologs added, in particular pentane-1,2-diol and/or hexane-1,2-diol; the amounts thereof may be from about 5% to about 15% by weight, the upper limit being limited only by the end product having sufficient strength.

### Pelletization Process

The process of the invention has the particular characteristic feature that 1,2-alkanediols having a smooth or near-smooth surface are produced.

In the first step, the 1,2-alkanediols are melted, i.e.—depending on the chain length—heated to about 60 to about 80° C. The melts thus obtained are then cooled while stirring.

This can be done for example in a stirred vessel with jacket cooling using a paddle stirrer. A supercooled (“prescratched”) melt in which seed crystals form is obtained—again depending on the chain length—at around 40 to 65° C. The cooling process is continued until the content of seed crystals is at least 1% by weight and preferably at least 5% by weight. Alternatively, seed crystals can also be externally added.

A further limiting factor is the viscosity, which is determined by the torque of the stirrer and should preferably be within a range from about 5 to about 15 Ncm and in particular about 8 to about 12 Ncm.

Once the prescratched melt has reached the required viscosity, it is dropletized for example onto a cooled surface, resulting in the formation of uniform and regular pellets.

After a rest period, which can be executed at ambient temperature and can be from about 1 to about 5 minutes, the pellets can be removed from the cooled surface, for example with a scraper.

Alternatively, the prescratched melt may also be solidified between two cooled surfaces, which may preferably be a distance of a few centimeters to a few millimeters apart.

This affords solid masses, but not pellets.

The temperature of the cooled surfaces can in each case be within a range from about 15 to about 22° C. and preferably between 18 and 20° C.

### 1,2-Alkanediols in Pelletized Form:

The present invention further provides:

Decane-1,2-diol in pelletized form, obtained by
(a) melting decane-1,2-diol at about 60° C.,
(b) gradually cooling the melt from step (a) to about 40 to 43° C., with stirring, until seed crystals separate out from the melt and the melt has reached a torque/viscosity of about 8 to about 12 Ncm,
(c) dropletizing the prescratched melt from step (b) onto a precooled surface having a temperature within a range from about 18 to about 20° C., and
(d) removing the pellets thus obtained from the precooled surface after a solidification time of about 1 to about 5 minutes.

Dodecane-1,2-diol in pelletized form, obtained by
(a) melting dodecane-1,2-diol at about 60° C.,
(b) gradually cooling the melt from step (a) to about 50 to 52° C., with stirring, until seed crystals separate out from the melt and the melt has reached a torque/viscosity of about 8 to about 12 Ncm,
(c) dropletizing the prescratched melt from step (b) onto a precooled surface having a temperature within a range from about 18 to about 20° C., and
(d) the pellets thus obtained after a solidification time of about 1 to about 5

Tetradecane-1,2-diol in pelletized form, obtained by
(a) melting tetradecane-1,2-diol at about 80° C.,
(b) gradually cooling the melt from step (a) to about 61 to 63° C., with stirring, until seed crystals separate out from the melt and the melt has reached a torque/viscosity of about 8 to about 12 Ncm,
(c) dropletizing the prescratched melt from step (b) onto a precooled surface having a temperature within a range from about 18 to about 20° C., and
(d) removing the pellets thus obtained from the precooled surface after a solidification time of about 1 to about 5 minutes.

### COMMERCIAL APPLICABILITY

The solid 1,2-alkanediols obtainable by the process of the invention can be packed for example in sacks and stored for practically any length of time without forming lumps and without abrasion and the formation of fine dust.

The pellets can be used for a variety of purposes, for example as constituents of cosmetic or pharmaceutical preparations.

### EXAMPLES

### Example 1

### Decane-1,2-diol

Production of Scratched Melts:

A melt (60° C.) of decane-1,2-diol (98%) was gradually cooled in a thermostated 250 mL jacketed vessel having a wall-sweeping paddle stirrer at a constant stirring speed (50 rpm). (The torque to be applied by the stirrer was recorded as a measure of the viscosity of the melt.) After reaching the melting point, the first crystals could be observed in the melt. At this point, seed crystals can optionally be added as a starter in order to accelerate the formation of the crystal bed. The temperature of the scratched mass was further cooled by 0.2° C./h, wherein an increase in the amount of crystals was observed. Once the desired viscosity of the melt had been reached, droplets were transferred by means of a pipette onto a steel surface cooled to 18-20° C. The time it took for the pellets to solidify was determined.

b) Pelletization Results:

| | |
|---|---|
| Temperature of the melt ° C. | 42.8-41.7 |
| Torque (viscosity) [Ncm] | 8.2-11.7 |
| Solidification time [sec] | 10-30 |
| Melting point [° C.] | 47.1-47.6 |
| Average height [mm] | 3.5-5.5 |
| Diameter [mm] | 11-16 |
| Individual weight [g] | 0.3-0.5 |

After a period of 1 minute, the pellets thus obtained were removed from the metal surface with the aid of a scraper and the consistency of the pellet was examined. The pellets were at this point in time completely solidified.

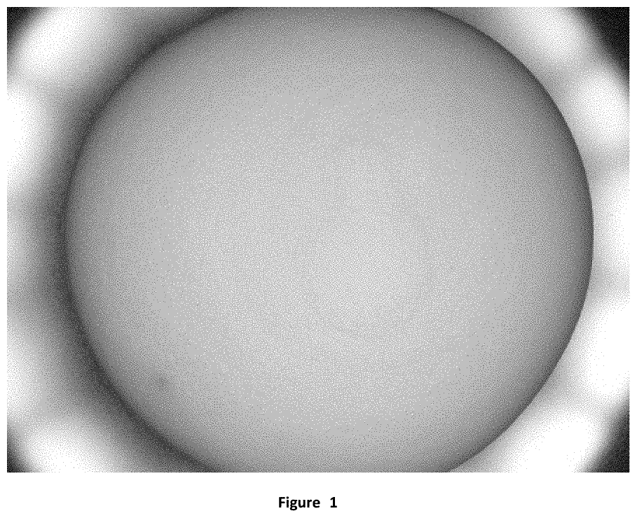

For storage tests, pellets were after removal from the metal surface stored in a bed height of 20 cm for 4 weeks at room temperature. After this time, the storage vessel was checked for lump formation and fines. The pellets produced according to example 1.1 were free-flowing and exhibited a very low degree of lump formation. No fines were observed in the storage vessel. FIG. 1 shows a pellet according to example 1.

### Example 2

### Double-Sided Cooling

A slurry produced by the above process having a temperature of 42.2° C. (9.5 Ncm) was introduced between two cooled metal surfaces a uniform distance apart (18° C., distance 2-5 mm). After a contact time of 1 minute, the upper surface was removed and the resulting solid was broken into flakes with a spatula. After removing fines, the material was used directly for storage tests. After a storage time of 4 weeks, the storage vessel was checked for lump formation and fines. The stored flakes were easy to split apart and had a minor content of fines (caused by mechanical stress during filling).

### Comparative Example C1

Melts of decane-1,2-diol (98.5%) having a defined temperature were dropletized by means of a pipette onto a temperature-controlled steel surface (19-21° C.). The time taken for the droplets/flakes thus produced to solidify was determined.

b) Pelletization Results:

| | |
|---|---|
| Melt temperature 45° C./50° C./60° C. | 1.5-2.0-2.5 |
| Melting point [° C.] | 47.1-47.6 |
| Average height [mm] | 3.0-3.5 |
| Diameter [mm] | 9-11 |
| Individual weight [g] | 0.2-0.3 |

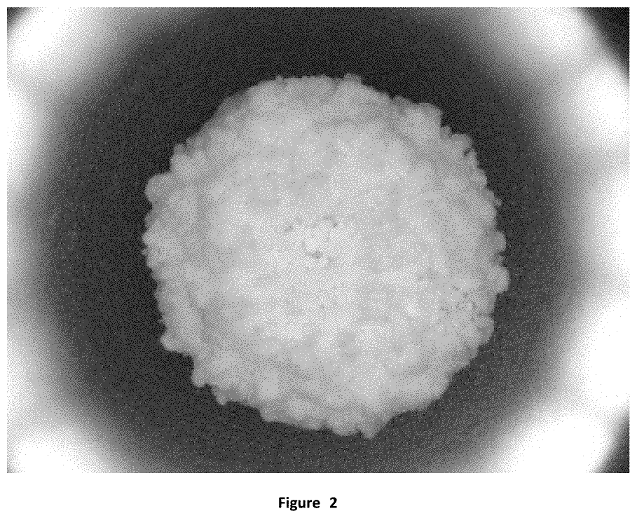

The droplets applied to the cooled surface showed a greater degree of spread, resulting in broader, flatter, and less raised solids. (lower height, greater base surface area). In all cases solids were obtained that had a rough, uneven surface with bubble-like inclusions. For storage tests, pellets were after removal from the metal surface stored in a bed height of approx. 20 cm for 4 weeks at room temperature. After this time, the storage vessel was checked for lump formation and fines. The pellets produced according to example 2 were not free-flowing and exhibited a high degree of lump formation. Fines caused by the delicate, rough surface were present in the storage vessel. These fines contribute, in addition to the rough surface of the pellets, to the formation of agglomerates/lumps in the pellets. FIG. 2 shows a pellet according to comparative example C1.

### Example 3

### Dodecane-1,2-diol

a) Production of Scratched Melts

A melt (60° C.) of dodecane-1,2-diol (99%) was gradually cooled in a thermostated 250 mL jacketed vessel having a wall-sweeping paddle stirrer at a constant stirring speed (50 rpm). (The torque to be applied by the stirrer was recorded as a measure of the viscosity of the melt.) After reaching the melting point, the first crystals could be observed in the melt. At this point, seed crystals can optionally be added as a starter in order to accelerate the formation of the crystal bed. The temperature of the scratched mass was further cooled by 0.2° C./h, wherein an increase in the amount of crystals was observed. Once the desired viscosity of the melt had been reached, droplets were transferred by means of a pipette onto a steel surface cooled to 18-20° C. The time it took for the pellets to solidify was determined.

b) Pelletization Results:

| | |
|---|---|
| Temperature of the melt [° C.] | 52.0-51.5 |
| Torque (viscosity) [Ncm] | 8.8-9.8 |
| Solidification time [sec] | 20-30 |
| Melting point [° C.] | 59.1-59.5 |
| Average height [mm] | 3.5-5.0 |
| Diameter [mm] | 10-15 |
| Individual weight [g] | 0.3-0.4 |

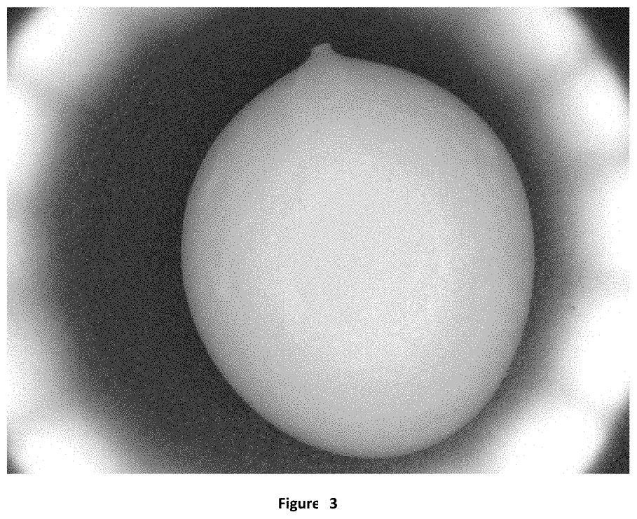

After a period of 1 minute, the pellets thus obtained were removed from the metal surface with the aid of a scraper and the consistency of the pellet was examined. The pellets were at this point in time completely solidified (i.e. they had no soft/unsolidified portions). FIG. 3 shows a pellet according to example 3.

### Comparative Example C2

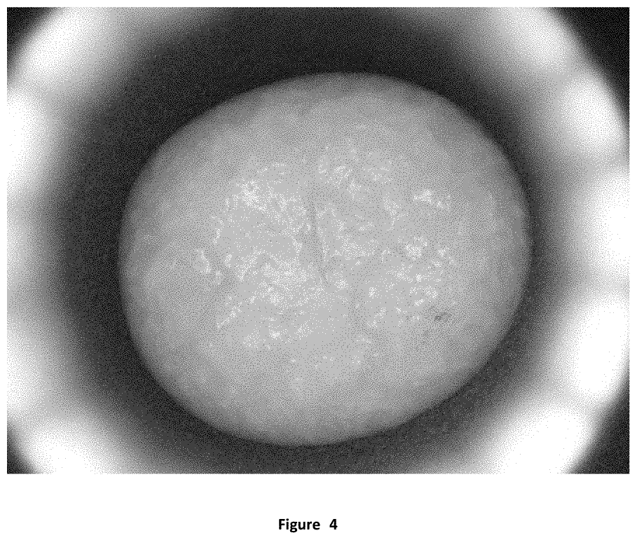

A melt of dodecane-1,2-diol (99%) was dropletized at 60° C. by means of a pipette onto a temperature-controlled steel surface (18-20° C.). After a period of 1 min, the consistency of the pellets was examined. The pellets thus produced were not completely hardened and had soft inclusions. The surfaces of the pellets thus produced were rough and uneven. Melting point of the pellets obtained: 59.0° C. FIG. 4 shows a pellet according to comparative example C2.

### Example 4

### Production of tetradecane-1,2-diol

a) Production of Scratched Melts:

A melt (80° C.) of tetradecane-1,2-diol (97%) was gradually cooled in a thermostated 250 mL jacketed vessel having a wall-sweeping paddle stirrer at a constant stirring speed (50 rpm). (The torque to be applied by the stirrer was recorded as a measure of the viscosity of the melt.) After reaching the melting point, the first crystals could be observed in the melt. At this point, seed crystals can optionally be added as a starter in order to accelerate the formation of the crystal bed. The temperature of the scratched mass was further cooled by 0.2° C./h, wherein an increase in the amount of crystals was observed. Once the desired viscosity of the melt had been reached, droplets were transferred by means of a pipette onto a steel surface cooled to 18-20° C. The time it took for the pellets to solidify was determined.

b) Pelletization Results:

| | |
|---|---|
| Temperature of the melt [° C.] | 62.0 |
| Torque (viscosity) [Ncm] | 8.7-11.5 |
| Solidification time [sec] | <10 |
| Melting point [° C.] | 66.5 |
| Average height [mm] | 3.5-4.5 |
| Diameter [mm] | 13.5-15.5 |
| Individual weight [g] | 0.5-0.6 |

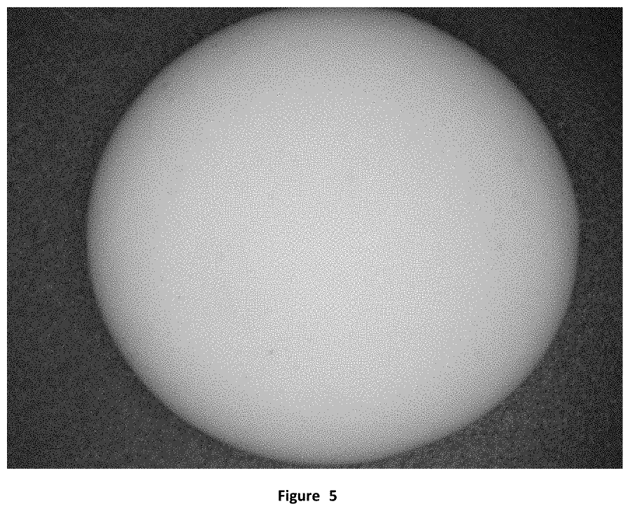

After a period of 1 minute, the pellets thus obtained were removed from the metal surface with the aid of a scraper and the consistency of the pellet was examined. The pellets were at this point in time completely solidified. FIG. 5 shows a pellet according to example 4.

### Comparative Example C3

A melt of tetradecane-1,2-diol (97%) was dropletized at 80° C. by means of a pipette onto a temperature-controlled steel surface (18-21° C.). After a period of 1 min, the consistency of the pellets was examined. The pellets thus produced were not completely hardened and had a few soft inclusions. The surfaces of the pellets thus produced were rough and uneven. Melting point of the pellets obtained: 64.3° C.

1. A process for producing 1,2-alkanediols in a solid dosing form, comprising the following steps:
(a) providing a melt of at least one 1,2-alkanediol;
(b) cooling the melt from step (a) to a temperature below the melting point of the 1,2-alkanediol or of the 1,2-alkanediol mixture to obtain a prescratched melt consisting of a supercooled 1,2-alkanediol or a supercooled 1,2-alkanediol mixture having a content of 1,2-alkanediol seed crystals;
(c) contacting the mixture from step (b) with a cooled surface to obtain 1,2-alkanediols or 1,2-alkanediol mixtures in crystalline form.

2. The process of claim 1, wherein the at least one alkanediol is selected from 1,2-alkanediols having 8 to 14 carbon atoms.

3. The process of claim 1 wherein the at least one 1,2-alkanediol is selected from the group consisting of octane-1,2-diol, decane-1,2-diol, dodecane-1,2-diol, and tetradecane-1,2-diol.

4. The process of claim 1, wherein the at least one 1,2-alkanediol is selected from technical grade 1,2-alkanediols having a 1,2-alkanediol content of at least 92% by weight.

5. The process of claim 1, wherein the 1,2-alkanediols or 1,2-alkanediol mixtures in crystalline form produced in step (c) are 1,2-alkanediols having a smooth or near-smooth surface.

6. The process of claim 1, wherein the melts are stirred while cooled in step (b).

7. The process of claim 1, where in step (b) the melts are cooled until the proportion of seed crystals in the prescratched melt is at least 1% by weight.

8. The process of claim 1, wherein additional seed crystals are added to the prescratched melt of step (b).

9. The process of claim 1, wherein the prescratched melt of step (b) is cooled with stirring until it has a torque/viscosity within a range of from about 5 to about 15 Ncm.

10. The process of claim 1, where in step (c) the prescratched melt is dropletized onto the cooled surface.

11. The process of claim 1, wherein in step (c) the contacting of the mixture from step (b) with a cooled surface comprises solidifying the prescratched melt between two cooled surfaces.

12. The process of claim 1, wherein in step (c) the cooled surface has a temperature within a range of from about 15 to about 22° C.

13. Decane-1,2-diol in pelletized form, obtained by a process comprising:
(a) melting decane-1,2-diol at about 60° C.,
(b) gradually cooling the melt from step (a) to about 40 to 43° C., with stirring, until seed crystals separate out from the melt and the melt has reached a torque/viscosity of about 8 to about 12 Ncm,
(c) dropletizing the prescratched melt from step (b) onto a precooled surface having a temperature within a range of from about 18 to about 20° C., and
(d) removing the pellets thus obtained from the precooled surface after a solidification time of from about 1 to about 5 minutes.

14. Dodecane-1,2-diol in pelletized form, obtained by a process comprising:
(a) melting dodecane-1,2-diol at about 60° C.,
(b) gradually cooling the melt from step (a) to about 50 to 52° C., with stirring, until seed crystals separate out from the melt and the melt has reached a torque/viscosity of about 8 to about 12 Ncm,
(c) dropletizing the prescratched melt from step (b) onto a precooled surface having a temperature within a range of from about 18 to about 20° C., and
(d) removing the pellets thus obtained from the precooled surface after a solidification time of from about 1 to about 5 minutes.

15. Tetradecane-1,2-diol in pelletized form, obtained by a process comprising:
(a) melting tetradecane-1,2-diol at about 80° C.,
(b) gradually cooling the melt from step (a) to about 61 to 63° C., with stirring, until seed crystals separate out from the melt and the melt has reached a torque/viscosity of about 8 to about 12 Ncm,
(c) dropletizing the prescratched melt from step (b) onto a precooled surface having a temperature within a range of from about 18 to about 20° C., and
(d) removing the pellets thus obtained from the precooled surface after a solidification time of from about 1 to about 5 minutes.

16. The process of claim 1, wherein in step (a) the at least one 1,2-alkanediol is at least two alkanediols selected from the group consisting of octane-1,2-diol, decane-1,2-diol, dodecane-1,2-diol, and tetradecane-1,2-diol.

17. The process of claim 4, wherein the at least one 1,2-alkanediol is selected from technical grade 1,2-alkanediols having a 1,2-alkanediol content of at least 95% by weight.
